(19) United States
(12) Patent Application Publication
(10) Pub. No.: US 2026/0069628 A1
(43) Pub. Date: Mar. 12, 2026

(54) TREATMENT OF PSORIASIS, PSORIATIC ARTHRITIS AND DERMATITIS

(71) Applicant: PARADIGM BIOPHARMACEUTICALS LTD, Melbourne, Victoria (AU)

(72) Inventors: Paul Rennie, Melbourne, Victoria (AU); Ravi Krishnan, Melbourne, Victoria (AU); Philip Bloom, Box Hill, Victoria (AU)

(21) Appl. No.: 19/108,856

(22) PCT Filed: Sep. 6, 2023
(86) PCT No.: PCT/AU2023/050861

§ 371 Date: Mar. 5, 2025

(30) Foreign Application Priority Data
Sep. 6, 2022 (AU) 2022902567

(51) Int. Cl. A61K 31/737; A61P 17/06

(52) U.S. Cl. CPC A61K 31/737; A61P 17/06

(57) ABSTRACT

The disclosure of the present invention relates to methods, uses and compositions for the treatment of psoriasis, psoriatic arthritis and dermatitis with pentosan polysulfate, or a pharmaceutically acceptable salt thereof.

### TECHNICAL FIELD

The disclosure of the present invention relates to methods, uses and compositions for the treatment of psoriasis, psoriatic arthritis and dermatitis with pentosan polysulfate, or a pharmaceutically acceptable salt thereof.

### BACKGROUND

Pentosan polysulfate (PPS) is a weaker anticoagulant than heparin but has been used post-surgically and prophylactically as a thrombolytic agent. Typically, PPS is used as the sodium salt (NaPPS). When given via the oral and intravesical routes, NaPPS has been used for the treatment of interstitial cystitis. Indeed, NaPPS is the active agent in the drug ELMIRON® which is currently prescribed for interstitial cystitis. The potential use of PPS and salts thereof in treatment of inflammatory conditions such as asthma, allergic rhinitis, and/or chronic obstructive pulmonary disease (COPD) has also been described, as has its use in the treatment of osteoporosis and bone marrow edema.

Dermatitis is an inflammation of the skin. The condition can be caused by genetics, an overactive immune system, infections, allergies, irritating substances and other factors. There are different types of dermatitis, such as atopic dermatitis, contact dermatitis, diaper dermatitis, dyshidrotic dermatitis, neurodermatitis, nummular dermatitis, perioral/periorificial dermatitis, seborrheic dermatitis and stasis dermatitis. Treatment regimens are focused on symptom control and include corticosteroid creams, gels or ointments, oral steroids such as prednisone, injectable steroids such as dupilumab, phototherapy and immunosuppressive medications.

Psoriasis is a chronic autoimmune condition that can affect the skin, nails and joints. It affects approximately 2% of the world's population. The condition causes the rapid growth of cells on the skin which causes thick, scaly plaques. There are five different types of psoriasis: plaque psoriasis, guttate (droplet) psoriasis, inverse psoriasis, pustular psoriasis and erythrodermic psoriasis.

Current treatment approaches of psoriasis include topical treatments, light therapy, systemic medications and combinations thereof. Topical treatments include corticosteroids, vitamin D analogues, retinoids, calcineurin inhibitors, salicylic acid, coal tar and anthralin. Light therapy treatments include sunlight therapy, UVB broadband and UVB narrowband therapy, psoralen plus ultraviolet A (PUVA) and Excimer laser. Systemic psoriasis treatments include steroids, retinoids, methotrexate, cyclosporine, biologics, hydroxyurea and combinations thereof.

Psoriasis can cause a form of arthritis, also known as psoriatic arthritis. Approximately 23% of psoriasis patients develop psoriatic arthritis within 10 years of the psoriasis diagnosis.

Psoriatic arthritis causes swollen, painful joints. Treatment approaches include non-steroidal anti-inflammatory drugs (NSAIDs), such as ibuprofen, etoricoxib, etodolac and naproxen, steroids (injections and tablets) and disease-modifying anti-rheumatic drugs (DMARDs), such as methotrexate, sulfasalazine and leflunomide.

Since there are currently no known cures for psoriasis, dermatitis and/or psoriatic arthritis, all treatments relate to symptom management. Most treatments have to be taken or applied frequently for a prolonged period of time and many have serious side effects. It is, therefore, desirable to provide improved treatments for such conditions.

### SUMMARY

In a first aspect, the disclosure resides in a method of treating a condition in a subject selected from the group consisting of psoriasis, psoriatic arthritis and dermatitis comprising administering an effective amount of pentosan polysulfate, or a pharmaceutically acceptable salt thereof, to the subject.

In a second aspect, the disclosure resides in a use of pentosan polysulfate, or a pharmaceutically acceptable salt thereof, in the manufacture of a medicament for the treatment of a condition selected from the group consisting of psoriasis, psoriatic arthritis and dermatitis.

In a third aspect, the disclosure resides in a use of pentosan polysulfate, or a pharmaceutically acceptable salt thereof, for the treatment of a condition selected from the group consisting of psoriasis, psoriatic arthritis and dermatitis.

In a fourth aspect, the disclosure resides in pentosan polysulfate, or a pharmaceutically acceptable salt thereof, for use in the treatment of a condition selected from the group consisting of psoriasis, psoriatic arthritis and dermatitis.

In a fifth aspect, the disclosure resides in a composition comprising pentosan polysulfate, or a pharmaceutically acceptable salt thereof, for the treatment of a condition selected from the group consisting of psoriasis, psoriatic arthritis and dermatitis.

In embodiments of the first to fifth aspect, the condition is psoriasis.

In embodiments, the psoriasis may be selected from the group consisting of plaque psoriasis, guttate psoriasis, pustular psoriasis, inverse psoriasis and erythrodermic psoriasis.

In embodiments of the first to fifth aspect, the condition is psoriatic arthritis.

In embodiments, the psoriatic arthritis is a complication of, or is associated with, the psoriasis in the subject.

In embodiments of the first to fifth aspect, the condition is dermatitis.

In embodiments, the dermatitis may be selected from the group consisting of contact dermatitis, atopic dermatitis, diaper dermatitis, dyshidrotic dermatitis, neurodermatitis, nummular dermatitis, perioral/periorificial dermatitis, seborrheic dermatitis and stasis dermatitis.

In an embodiment, the dermatitis is selected from the group consisting of contact dermatitis, atopic dermatitis and diaper dermatitis.

In an embodiment, the condition is contact dermatitis. In another embodiment, the condition is atopic dermatitis. In another embodiment, the condition is diaper dermatitis.

In embodiments of the first to fifth aspect, the pentosan polysulfate, or a pharmaceutically acceptable salt thereof, is administered by subcutaneous injection, intravenous injection, oral administration or topical administration.

In embodiments of the first to fifth aspect, if the pentosan polysulfate, or a pharmaceutically acceptable salt thereof, is administered by topical administration, the composition is formulated as a topical composition.

In an embodiment, the topical composition is formulated as a cream, ointment, gel, lotion, foam, powder, aerosol, spray or liquid solution.

In embodiments of the first to fifth aspect, if the treatment is administered by subcutaneous or intravenous injection, the composition is formulated as an injectable composition.

In embodiments of the first to fifth aspect, if the treatment is administered orally, the composition is formulated as an oral composition.

In embodiments, pentosan polysulfate, or a pharmaceutically acceptable salt thereof, may be co-administered with drugs that are commonly used to treat psoriasis, psoriatic arthritis or dermatitis.

### DESCRIPTION OF EMBODIMENTS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a cell” includes populations of a plurality of cells.

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 2, from 1 to 3, from 1 to 4, from 2 to 3, from 2 to 4, from 2 to 5, from 3 to 4 etc., as well as individual and partial numbers within the recited range, for example, 1, 2, 3, 4, and 5. This applies regardless of the breadth of the disclosed range. Where specific values are required, these will be indicated in the specification.

As used herein, the term “about” refers to a range of ±10% of the specified value or a range associated with the experimental error known to the skilled addressee in measuring the specified value, whichever is the greater.

The term “acceptable excipient” includes excipients or agents such as solvents, diluents, dispersion media, coatings, antibacterial and antifungal agents, isotonic and absorption delaying agents and the like that are physiologically compatible and are not deleterious to a compound as described herein or use thereof. The use of such carriers and agents to prepare compositions of pharmaceutically active substances is well known in the art. For example as taught in Remington: The Science and Practice of Pharmacy, 21st Edition.

The term “pharmaceutically acceptable salts” include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate and the like; amino acid salts such as arginate, asparaginate, glutamate and the like; metal salts such as sodium salt, potassium salt, cesium salt and the like; alkaline earth metals such as calcium salt, magnesium salt and the like; and organic amine salts such as triethylamine salt, pyridine salt, picoline salt, ethanolamine salt, triethanolamine salt, discyclohexylamine salt, N,N′-dibenzylethylenediamine salt and the like.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of acceptable salts and their formation is known to those skilled in the art and is as described in general texts. For example, “Handbook of Pharmaceutical salts” P. H. Stahl, C. G. Wermuth, 1st edition, 2002,

The terms “administration of” and/or “administering a”, in relation to treatments described herein, should be understood to mean providing PPS, or a pharmaceutically acceptable salt thereof, to the individual in need of treatment.

The term “composition” as used herein is intended to encompass a product comprising the specified ingredients in the specified amounts, as well as any product which results, directly or indirectly, from combination of the specified ingredients in the specified amounts.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

This disclosure is directed to treatment of human subjects. The treatment of a “human subject” may also be referred to the treatment of a “patient” or an “individual”. A human subject is one who has presented a clinical manifestation of a particular symptom or symptoms suggesting the need for treatment, is being treated for a condition, or who has been diagnosed with a condition to be treated or who is suspected of having a condition to be treated. Herein, the condition requiring treatment is one selected from the group consisting of psoriasis, psoriatic arthritis and dermatitis.

As used herein, the terms “treating”, “treat” or “treatment” and variations thereof, refer to clinical intervention designed to alter the natural course of the subject during the course of clinical pathology. Desirable effects of treatment include decreasing the rate of disease progression, ameliorating or palliating the disease state, and remission or improved prognosis. A subject is successfully “treated”, for example, if one or more of the above treatment outcomes is achieved.

An “effective amount” encompasses a “therapeutically effective” amount which refers to at least the minimum concentration or amount required to effect a measurable improvement of a particular disease. An effective amount herein may vary according to factors such as the disease state, age, sex, and weight of the patient, and the ability of the PPS to elicit a desired response in the individual. An effective amount is also one in which any toxic or detrimental effects of the PPS are outweighed by the therapeutically beneficial effects. An “effective amount” also encompasses a “prophylactically effective” amount which refers to the amount of drug or the rate of drug administration needed to produce the desired preventive result.

Unless specified otherwise, pentosan polysulfate (PPS), as referred to herein, refers to a complex mixture of highly sulfated xylan with an average molecular weight of between about 3000 Da to about 7000 Da, or between about 4000 Da to about 7000 Da, preferably between about 4000 Da to about 6000 Da. The PPS may have a degree of sulfation of from 1.5 to 2.3 sulfate esters per xylose units, preferably between 1.9 to 2.2 and more preferably about 2.2. The PPS may have a sulfur content from 16 to 19% (w/w), preferably about 19%.

The PPS, or pharmaceutically acceptable salt thereof, for use in the present disclosure may be unfractionated. That is, the PPS, or pharmaceutically acceptable salt thereof, has not been subject to post-synthesis or post-manufacture fractionation to provide for fractions with narrower molecular weight distributions. Put another way, it is an advantage of the present treatment approach that commercially available PPS (such as that available from Bene-PharmaChem GmbH & Co KG, Geretsried, Germany and approved by the FDA as the interstitial cystitis treatment ELMIRON®) is suitable for use directly in the present treatment approaches without fractionation or chemical modification. A very narrow molecular weight distribution or polydispersity index (PDI) is not required.

The measured PDI of a PPS sample can vary depending on the analytical approach taken with two main approaches being taken. When measured using a size-exclusion chromatography (SEC)-Refractive Index (RI) approach as described in the Examples, the PPS, or pharmaceutically salt thereof, may have a polydispersity index from about 1.8 to about 2.4, preferably from about 1.9 to about 2.3, more preferably from about 1.95 to 2.2. In embodiments, the PPS may have a PDI of about 2.0, about 2.1 or about 2.2, ±0.2 standard deviation. Preferably, the PPS has a PDI of 2.1 with ±0.2 standard deviation.

When measured using a size-exclusion chromatography (SEC)-Triple Detection (TD) approach as described in the Examples, the PPS, or pharmaceutically salt thereof, may have a polydispersity index of greater than 1.5 to about 1.8, preferably from about 1.52 to about 1.75, more preferably from about 1.54 to 1.72. In embodiments, the PPS may have a PDI of about 1.55, about 1.60, about 1.65 or about 1.70.

PPS consists of repeating linear units of 1-4 linked ß-D-xylopyranose which are laterally substituted with 4-O-methylglucopyranosyluronic acid units to the 2-position of the main chain at about every tenth xylopyranose unit on average. Prior art reports that the number of xylose units in a typical PPS mixture may vary from 1 to 50, such as from 6 to 30. It will be understood by a person of skill in the art that all variations within this definition, for example regarding chain length, percentage of sulfation, average molecular weight, substitution ratio etc., fall within the scope of a PPS as referred to throughout the present disclosure. So, depending on the composition of the natural raw material, some xylose may be substituted by other sugars and some xylose units may be esterified with an acetyl group instead of sulfate. Preparations of PPS that can be utilised are, for example, commercially available from Bene-PharmaChem GmbH & Co KG, Geretsried, Germany.

Pentosan polysulfate according to the present disclosure may be provided as an alkali metal salt or alkaline earth metal salt, for example, comprising calcium or sodium salt, or transition metals such as copper and zinc and noble metals such as platinum. Accordingly, the particular complexing ions may be selected from the group consisting of the alkali metals, e.g. Na⁺ and K⁺, alkaline earth metals, e.g. Ca²⁺, Zn²⁺, Mg²⁺, Ba²⁺, as well as Ag⁺, Pb²⁺, Cu²⁺, Au²⁺, Pd²⁺, Pd⁴⁺, Pd⁴⁺, Pd²⁺, trivalent metal ions, and quaternary ammonium compound complexes. Examples of the latter compound include pyridinium chloride, tetraalkyl ammonium chloride, chorine chloride, cetylpyridinium chloride, N-cetyl-N,N,N-trialkyl ammonium chloride and their derivatives. Preparation of the polysulfate polysaccharide-metal complexes is described in detail in U.S. Pat. No. 5,668,116; see also U.S. Patent Publication No. 2009/0111771 to Cullis-hill et al., which are hereby incorporated by reference in their entirety.

In some embodiments, the pentosan polysulfate is present as a pharmaceutically acceptable salt. The pharmaceutically acceptable salt may be selected from a sodium salt of pentosan polysulfate (NaPPS), a magnesium salt of pentosan polysulfate (MgPPS), a calcium salt of pentosan polysulfate (CaPPS), and a zinc salt of pentosan polysulfate (ZnPPS). In preferred embodiments, the pentosan polysulfate is present as a sodium salt of pentosan polysulfate (NaPPS).

It will be recognized by persons skilled in the art, that PPS and PPS compositions suitable for administration by a variety of routes may be formulated by reference to standard textbooks in this field. For example, Remington: The Science and Practice of Pharmacy, 21st Edition. Compositions may be formulated from PPS and pharmaceutically acceptable salts thereof for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions).

In some embodiments, the pentosan polysulfate, or a pharmaceutically acceptable salt thereof, or composition comprising the pentosan polysulfate or pharmaceutically acceptable salt thereof, is administered by or formulated for administration by: subcutaneous injection, intravenous injection, oral administration or topical administration. In embodiments where the pentosan polysulfate or pharmaceutically acceptable salt thereof is administered by subcutaneous or intravenous injection, the composition may be formulated as an injectable composition. In embodiments where the pentosan polysulfate or pharmaceutically acceptable salt thereof is administered orally, the composition may be formulated as an oral composition, for example tablets, troches, lozenges, aqueous or oily suspensions, dispersible powders or granules, emulsions, hard or soft capsules, or syrups or elixirs. In embodiments where the pentosan polysulfate or pharmaceutically acceptable salt thereof is administered by topical administration, the composition may be formulated as a topical composition, for example formulated as a cream, ointment, gel, lotion, foam, powder, aerosol, spray or liquid solution. The formulations may be present in unit or multi-dose containers such as sealed ampoules or vials.

The present disclosure describes the use of pentosan polysulfate, or a pharmaceutically acceptable salt thereof, to treat psoriasis, psoriatic arthritis and/or dermatitis in a patient.

It has been surprisingly found that pentosan polysulfate, or a pharmaceutically acceptable salt thereof, alleviates the symptoms associated with psoriasis. Psoriasis is a chronic autoimmune condition that can affect the skin, nails and joints. It affects approximately 2% of the world's population. The condition causes the rapid growth of cells on the skin which causes thick, scaly plaques.

There are five different types of psoriasis: plaque psoriasis, guttate (droplet) psoriasis, inverse psoriasis, pustular psoriasis and erythrodermic psoriasis. Plaque psoriasis, also known as psoriasis vulgaris, is the most common with about 85-90% of psoriasis patients being affected. It usually shows as dry, itchy, inflamed and raised skin patches that are covered in silvery-white scales.

Guttate (droplet) psoriasis is characterised by scaly, teardrop-shaped papules and plaques. Guttate psoriasis is more common in children and adolescents than in adults and is usually triggered by a streptococcal infection. About one third of children and adolescents who suffered from guttate psoriasis will develop plaque psoriasis later in life.

Inverse psoriasis is characterised by smooth, moist, dark-red and inflamed patches. It mainly affects the skin folds in the body, such as the groin, buttocks and breasts. The condition can worsen with friction and sweating.

Pustular psoriasis is characterised by white coalescing pustules. This type of psoriasis most commonly affects the palms of the hands, the soles of the feet and the fingers and toes. The condition can be acute or chronic.

Erythrodermic psoriasis is a rare but very serious complication of psoriasis. People suffering from erythrodermic psoriasis experience a peeling rash and plaques that cover almost the entire body. This condition can be acute or chronic.

There is currently no cure for psoriasis. Current treatment approaches relate to symptom management and include topical treatments, light therapy, systemic medications and combinations thereof. Topical treatments include corticosteroids, vitamin D analogues, retinoids, calcineurin inhibitors, salicylic acid, coal tar and anthralin. Light therapy treatments include sunlight therapy, UVB broadband and UVB narrowband therapy, psoralen plus ultraviolet A (PUVA) and Excimer laser. Systemic psoriasis treatments include steroids, retinoids, methotrexate, cyclosporine, biologics, hydroxyurea and combinations thereof.

Psoriatic arthritis causes swollen, painful joints. Treatment approaches include non-steroidal anti-inflammatory drugs (NSAIDs), such as ibuprofen, etoricoxib, etodolac and naproxen, steroids (injections and tablets) and disease-modifying anti-rheumatic drugs (DMARDs), such as methotrexate, sulfasalazine and leflunomide.

Given the relationship between the symptoms of psoriasis and psoriatic arthritis and dermatitis, it can be reasonably expected that pentosan polysulfate, or a pharmaceutically acceptable salt thereof, can be also used to successfully treat the symptoms of dermatitis, having been demonstrated in the present Examples to treat psoriasis.

Dermatitis is an inflammation of the skin. The condition can be caused by genetics, an overactive immune system, infections, allergies, irritating substances and other factors. Common symptoms of dermatitis are itchiness, dry skin, rashes on swollen skin, blisters that can be oozing and crusting, flaking skin and thickened skin. Dermatitis can be painful for many patients. There are different types of dermatitis, such as atopic dermatitis, contact dermatitis, diaper dermatitis, dyshidrotic dermatitis, neurodermatitis, nummular dermatitis, perioral/periorificial dermatitis, seborrheic dermatitis and stasis dermatitis.

Atopic dermatitis, also known as eczema, is a chronic condition that affects around 2-3% of adults and 25% of children. Symptoms include itchy, red rashes, dry skin, flaky or scaly patches.

Contact dermatitis is a reaction to an allergen or irritant that leads to a painful or itchy skin rash. The skin rash that develops can be red, swollen, blistering, burning or stinging, flaky, itchy and painful.

A sub-category to irritant contact dermatitis is diaper dermatitis, commonly referred to as ‘nappy rash’. This condition usually affects infants aged 3 to 15 months of age. It can be caused by over-hydration of the skin, maceration and prolonged contact with urine and faeces. Symptoms include inflamed and itchy skin and sores in the diaper area (buttocks, thighs and genitals).

Since there is currently no known cure for dermatitis, treatment regimens are focused on symptom control. Treatments include corticosteroid creams, gels or ointments, oral steroids, such as prednisone, injectable steroids, such as dupilumab, phototherapy and immunosuppressive medications.

In a first aspect, the disclosure resides in a method of treating a condition in a subject selected from the group consisting of psoriasis, psoriatic arthritis and dermatitis comprising administering an effective amount of pentosan polysulfate, or a pharmaceutically acceptable salt thereof, to the subject.

In a second aspect, the disclosure resides in a use of pentosan polysulfate, or a pharmaceutically acceptable salt thereof, in the manufacture of a medicament for the treatment of a condition selected from the group consisting of psoriasis, psoriatic arthritis and dermatitis.

In a third aspect, the disclosure resides in a use of pentosan polysulfate, or a pharmaceutically acceptable salt thereof, for the treatment of a condition selected from the group consisting of psoriasis, psoriatic arthritis and dermatitis.

In a fourth aspect, the disclosure resides in pentosan polysulfate, or a pharmaceutically acceptable salt thereof, for use in the treatment of a condition selected from the group consisting of psoriasis, psoriatic arthritis and dermatitis.

In a fifth aspect, the disclosure resides in a pharmaceutical composition comprising pentosan polysulfate, or a pharmaceutically acceptable salt thereof, for the treatment of a condition selected from the group consisting of psoriasis, psoriatic arthritis and dermatitis.

In embodiments of the first to fifth aspect, the condition is psoriasis.

In embodiments, the psoriasis may be selected from the group consisting of plaque psoriasis, guttate psoriasis, pustular psoriasis, inverse psoriasis and erythrodermic psoriasis.

The severity and extent of psoriasis can be measured using the Psoriasis Area and Severity Index (PASI) score (See Clinical Review Report: Guselkumab (Tremfya): (Janssen Inc.): Indication: For the treatment of adult patients with moderate-to-severe plaque psoriasis who are candidates for systemic therapy or phototherapy [Internet]. Ottawa (ON): Canadian Agency for Drugs and Technologies in Health; 2018 Mar. Appendix 5, Validity of Outcome Measures, https://www.ncbi.nlm.nih.gov/books/NBK534046/;https://dermnetnz. org/topics/pasi-score, which are hereby incorporated in their entirety by reference).

The score ranges from 0 to 72. In general, a PASI score between 5 to 10 is considered moderate, and a score over 10 is considered to be severe.

In embodiments, the treatment with pentosan polysulfate, or a pharmaceutically acceptable salt thereof, reduces the psoriasis experienced by the subject to below 10 on the PASI score. In embodiments, the treatment with pentosan polysulfate, or a pharmaceutically acceptable salt thereof, reduces the psoriasis experienced by the subject to between about 5 to about 10 on the PASI score. In an embodiment, the treatment with pentosan polysulfate, or a pharmaceutically acceptable salt thereof, reduces the psoriasis to below 5, or below 4, or below 3, or below 2, or below 1.

In another embodiment, the treatment with pentosan polysulfate, or a pharmaceutically acceptable salt thereof, reduces the psoriasis experienced by the subject to about 5, or to about 4, or to about 3, or to about 2, or to about 1, on the PASI score.

In an embodiment, the subject is substantially free from psoriasis after the treatment with pentosan polysulfate, or a pharmaceutically acceptable salt thereof. The subject may be substantially free from the psoriasis in terms of clinical observations of the psoriasis for up to 3 months following the end of a course of treatment, or up to 6 months following the end of a course of treatment, or up to 9 months following the end of a course of treatment, or up to 12 months following the end of a course of treatment, or up to 24 months following the end of a course of treatment, or even beyond 24 months following the end of a course of treatment up to, for example, 3 or 4 years following the end of a course of treatment.

A 75% reduction in the PASI score (PASI 75) is the current benchmark for most clinical trials in psoriasis and the criterion for efficacy of new psoriasis treatments approved by the FDA.

In embodiments, the treatment with pentosan polysulfate, or a pharmaceutically acceptable salt thereof, results in a 50% to 100% reduction in the PASI score, or a 55% to 100% reduction, or a 60% to 100% reduction, or a 65% to 100% reduction, or a 70% to 100% reduction, or a 75% to 100% reduction, or a 80% to 100% reduction, or a 85% to 100% reduction, or a 90% to 100% reduction, or a 95% to 100% reduction, or a 96% to 100% reduction, or a 97% to 100% reduction, or a 98% to 100% reduction, or a 99% to 100% reduction.

In embodiments, the treatment with pentosan polysulfate, or a pharmaceutically acceptable salt thereof, results in a 70% to 100% reduction in the PASI score, or a 70% to 99% reduction, or a 70% to 98% reduction, or a 70% to 97% reduction, or a 70% to 96% reduction, or a 70% to 95% reduction, or a 70% to 94% reduction, or a 70% to 93% reduction, or a 70% to 92% reduction, or a 70% to 91% reduction, or a 70% to 90% reduction, or a 70% to 88% reduction, or a 70% to 85% reduction, or a 70% to 80% reduction.

In embodiments, the treatment with pentosan polysulfate, or a pharmaceutically acceptable salt thereof, results in a 75% to 100% reduction in the PASI score, or a 75% to 99% reduction, or a 75% to 98% reduction, or a 75% to 97% reduction, or a 75% to 96% reduction, or a 75% to 95% reduction, or a 75% to 94% reduction, or a 75% to 93% reduction, or a 75% to 92% reduction, or a 75% to 91% reduction, or a 75% to 90% reduction, or a 75% to 88% reduction, or a 75% to 85% reduction, or a 75% to 80% reduction.

In embodiments, the treatment with pentosan polysulfate, or a pharmaceutically acceptable salt thereof, results in a 80% to 100% reduction in the PASI score, or a 80% to 99% reduction, or a 80% to 98% reduction, or a 80% to 97% reduction, or a 80% to 96% reduction, or a 80% to 95% reduction, or a 80% to 94% reduction, or a 80% to 93% reduction, or a 80% to 92% reduction, or a 80% to 91% reduction, or a 80% to 90% reduction, or a 80% to 88% reduction, or a 80% to 85% reduction.

In embodiments, the treatment with pentosan polysulfate, or a pharmaceutically acceptable salt thereof, results in at least a 75% reduction of psoriasis in the PASI score, or at least a 80% reduction of psoriasis in the PASI score, or at least a 85% reduction of psoriasis in the PASI score, or at least a 90% reduction of psoriasis in the PASI score, or at least a 95% reduction of psoriasis in the PASI score, or at least a 96% reduction of psoriasis in the PASI score, or at least a 97% reduction of psoriasis in the PASI score, or at least a 98% reduction of psoriasis in the PASI score, or at least a 99% reduction of psoriasis in the PASI score.

In embodiments of the first to fifth aspect, the condition is psoriatic arthritis. Historically, there are five main types of psoriatic arthritis. Hence, in one embodiment, the psoriatic arthritis may be selected from the group consisting of asymmetric oligoarticular, symmetric polyarticular, arthritis mutilans, spondylitis, and distal interphalangeal (DIP) predominant.

Similarly to psoriasis, psoriatic arthritis can range from mild to severe. When less than four joints in the body are affected the psoriatic arthritis is referred to as asymmetric oligoarthritis. Asymmetric oligoarthritis is considered mild psoriatic arthritis. Prior art reports state that between 35 and 70% of subjects suffering from psoriatic arthritis have asymmetric oligoarticular psoriatic arthritis.

The psoriatic arthritis is classified as severe when more than five joints (symmetric polyarticular) are affected. Between 25% to 50% of patients have polyarticular psoriatic arthritis.

Spondylitis (also referred to as psoriatic spondylitis or spondyloarthritis) refers to inflammation of the joints of the spine. According to prior art reports, about 7 to 32% of the subjects suffering from psoriatic arthritis have spondylitis.

Arthritis mutilans is a severe form of psoriatic arthritis that may result in deformation and destruction if the finger, hand, wrist, and/or feet joints. Less than 5% of subjects suffer from arthritis mutilans.

While the foregoing classification is still used in the medical field, in more recent reports, medical professionals have started to classify psoriatic arthritis based on the different domains (or manifestations) that patients tend to experience the symptoms (See https://creakyjoints./g/about-arthritis/psoriatic-arthritis/psa-overview/psoriatic-arthritis-types/ which is hereby incorporated by reference). Therefore, in another embodiment, the psoriatic arthritis may be selected from the group consisting of peripheral arthritis, axial disease, enthesitis, dactylitis, skin psoriasis and nail lesions.

Peripheral arthritis tends to move from one joint to another, impacting the large joints of the arms and hands (elbows, wrists) and legs (knees, ankles). People with peripheral arthritis are more likely to develop inflamed toes or fingers and enthesitis (inflammation of the spot where tendons or ligaments attach to the bone).

Axial disease (also referred to as spondylitis or spondyloarthropathy) causes pain and stiffness in the joints of the spine and sacroiliac joints.

Enthesitis is inflammation of the enthesis, which is where tendons or ligaments are attached to the bone.

Dactylitis is characterised by the painful, red and hot swelling of an entire finger or toe.

Nail lesions include pitting of the nail, crumbling, spotted lunula (red spots in the white arch above the cuticle), and splinter hemorrhages (blood spots under the nail).

In embodiments, the psoriatic arthritis is a complication of, or is associated with, the psoriasis in the patient.

In embodiments of the first to fifth aspect, the condition is dermatitis.

In embodiments, the dermatitis may be selected from the group consisting of contact dermatitis, atopic dermatitis, diaper dermatitis, dyshidrotic dermatitis, neurodermatitis, nummular dermatitis, perioral/periorificial dermatitis, seborrheic dermatitis and stasis dermatitis.

In an embodiment, the dermatitis is selected from the group consisting of contact dermatitis, atopic dermatitis or diaper dermatitis.

In an embodiment, the condition is contact dermatitis. In another embodiment, the condition is atopic dermatitis. In another embodiment, the condition is diaper dermatitis.

The extent and severity of atopic dermatitis can be measured using the Eczema Area and Severity Index (EASI) score. The score takes into consideration: (i) the four different body regions: head and neck, trunk, upper limbs and lower limbs, (ii) the area of each body region that is affected expressed in a percentage value from 0% to 100%, and (iii) the severity on a scale from 0 (none) to 3 (severe) for each of the four regions, assessing redness, thickness, scratching and lichenification. The final score is between 0 and 72.

A score of 0 indicates clear or no eczema, 0.1 to 1.0 indicates almost clear, 1.1 to 7 indicates mild disease, 7.1 to 21 indicates moderate disease, 21.1 to 50 indicates severe disease, and greater than 51 indicates very severe disease.

In embodiments, the treatment with pentosan polysulfate, or a pharmaceutically acceptable salt thereof, may reduce the atopic dermatitis to below 10 on the EASI score, or below 9 on the EASI score, or below 8 on the EASI score, or below 7 on the EASI score. In embodiments, the treatment with pentosan polysulfate, or a pharmaceutically acceptable salt thereof, may reduce the atopic dermatitis to between about 5 to about 10 on the EASI score, or between about 5 to about 9 on the EASI score, or between about 5 to about 8 on the EASI score, or between about 5 to about 7 on the EASI score.

In an embodiment, the treatment with pentosan polysulfate, or a pharmaceutically acceptable salt thereof, may reduce the atopic dermatitis to below 5, or below 4, or below 3, or below 2, or below 1 on the EASI score.

In another embodiment, the treatment with pentosan polysulfate, or a pharmaceutically acceptable salt thereof, may reduce the atopic dermatitis to about 5, or to about 4, or to about 3, or to about 2, or to about 1, on the EASI score.

In an embodiment, the subject may be substantially free from atopic dermatitis after the treatment with pentosan polysulfate, or a pharmaceutically acceptable salt thereof.

In embodiments, the treatment with pentosan polysulfate, or a pharmaceutically acceptable salt thereof, may result in a 50% to 100% reduction in the EASI score, or a 55% to 100% reduction, or a 60% to 100% reduction, or a 65% to 100% reduction, or a 70% to 100% reduction, or a 75% to 100% reduction, or a 80% to 100% reduction, or a 85% to 100% reduction, or a 90% to 100% reduction, or a 95% to 100% reduction, or a 96% to 100% reduction, or a 97% to 100% reduction, or a 98% to 100% reduction, or a 99% to 100% reduction of atopic dermatitis.

In embodiments, the treatment with pentosan polysulfate, or a pharmaceutically acceptable salt thereof, may result in a 70% to 100% reduction in the EASI score, or a 70% to 99% reduction, or a 70% to 98% reduction, or a 70% to 97% reduction, or a 70% to 96% reduction, or a 70% to 95% reduction, or a 70% to 94% reduction, or a 70% to 93% reduction, or a 70% to 92% reduction, or a 70% to 91% reduction, or a 70% to 90% reduction, or a 70% to 88% reduction, or a 70% to 85% reduction, or a 70% to 80% reduction of atopic dermatitis.

In embodiments, the treatment with pentosan polysulfate, or a pharmaceutically acceptable salt thereof, may result in a 75% to 100% reduction in the EASI score, or a 75% to 99% reduction, or a 75% to 98% reduction, or a 75% to 97% reduction, or a 75% to 96% reduction, or a 75% to 95% reduction, or a 75% to 94% reduction, or a 75% to 93% reduction, or a 75% to 92% reduction, or a 75% to 91% reduction, or a 75% to 90% reduction, or a 75% to 88% reduction, or a 75% to 85% reduction, or a 75% to 80% reduction of atopic dermatitis.

In embodiments, the treatment with pentosan polysulfate, or a pharmaceutically acceptable salt thereof, may result in a 80% to 100% reduction in the EASI score, or a 80% to 99% reduction, or a 80% to 98% reduction, or a 80% to 97% reduction, or a 80% to 96% reduction, or a 80% to 95% reduction, or a 80% to 94% reduction, or a 80% to 93% reduction, or a 80% to 92% reduction, or a 80% to 91% reduction, or a 80% to 90% reduction, or a 80% to 88% reduction, or a 80% to 85% reduction of atopic dermatitis.

In embodiments, the treatment with pentosan polysulfate, or a pharmaceutically acceptable salt thereof, may result in at least a 75% reduction of atopic dermatitis in the EASI score, or at least a 80% reduction of atopic dermatitis in the EASI score, or at least a 85% reduction of atopic dermatitis in the EASI score, or at least a 90% reduction of atopic dermatitis in the EASI score, or at least a 95% reduction of atopic dermatitis in the EASI score, or at least a 96% reduction of atopic dermatitis in the EASI score, or at least a 97% reduction of atopic dermatitis in the EASI score, or at least a 98% reduction of atopic dermatitis in the EASI score, or at least a 99% reduction of atopic dermatitis in the EASI score.

In embodiments of the first to fifth aspect, the pentosan polysulfate, or a pharmaceutically acceptable salt thereof, is administered by subcutaneous injection, intravenous injection, oral or topical administration.

In embodiments of the first to fifth aspects, if the pentosan polysulfate, or a pharmaceutically acceptable salt thereof, is administered by topical administration, the pharmaceutical composition is formulated as a topical composition.

In an embodiment, the topical composition is formulated as a cream, ointment, gel, lotion, foam, powder, aerosol, spray or liquid solution. The formulations may be present in unit or multi-dose containers such as sealed ampoules or vials.

A typical base for a topical composition containing pentosan polysulfate, or a pharmaceutically acceptable salt thereof, as the active pharmaceutical ingredient may comprise water, an optional penetration enhancer, and a topical carrier.

In embodiments, the amount of water incorporated in the present formulation is less than or equal to 30 weight percent and more preferably less than or equal to 25 weight percent. The amount of water used in the formulation of present invention is in the range of 2 weight percent to 30 weight percent of the formulation. In an embodiment, the amount of water in the formulation of the present invention ranges from 2 weight percent to 25 weight percent of the formulation. In another embodiment, the amount of water used in the formulation of the present invention is less than or equal to 10 weight percent of the formulation.

The penetration enhancer(s) used in accordance with the present disclosure are lower chain alcohol(s) with a carbon chain length ranging from C1 to C5 or mixtures thereof. In embodiments, the penetration enhancers of the present disclosure are selected from the group consisting of ethanol, isopropanol and their like, or mixtures thereof. The formulations of the present disclosure avoid the use of such penetration enhancers in high proportion and thereby avoid adverse effects on the skin such as dehydration and irritation of skin in case of alcohols. The said penetration enhancer(s) of the present disclosure is used in the range of 10 to 30 weight percent of the formulation. In the preferred embodiments, the penetration enhancer of the present disclosure is used in the range of 10 to 20 weight percent. In an embodiment, the penetration enhancer(s) is used in the amount of 10 weight percent of the formulation.

The formulations of present disclosure use one or more topical carrier(s) as the principal vehicle of the topical formulations. The said topical carrier used for the formulations of the present invention can be selected from a group comprising propylene glycol, glycerol, glycofurol, poly ethylene glycols (e.g. PEG400, PEG600 and the like), methylparaben, alginates, glyceryl stearate, PEG-100 stearate, cetostearyl alcohol, propylparaben, butylparaben, sorbitols, myristyl alcohol, polyethoxylated anhydrosorbitol monostearate (TWEEN), white soft paraffin (VASELINE), triethanolamine, aloe vera extract, lanolin, cocoa butter or mixtures thereof. Suitable topical carriers with different viscosities and hand-feel are well known to the skilled artisan. The said carrier for the topical formulations of the present disclosure is incorporated in an amount not less than 45% weight percent, preferably not less than 50 weight percent of the formulation.

Pentosan polysulfate, or a pharmaceutically acceptable salt thereof, is dispersed within the pharmaceutically acceptable carrier in therapeutically effective amounts to treat psoriasis and/or psoriatic arthritis and/or dermatitis. In embodiments, the pentosan polysulfate, or a pharmaceutically acceptable salt thereof, is present (per gram total weight) in an amount of about 0.01 weight percent to about 15 weight percent, or about 0.1 weight percent to about 10 weight percent, or about 1 weight percent to about 8 weight percent, or about 2 weight percent to about 7 weight percent.

In embodiments, the topical pharmaceutical formulation of the present disclosure may be formulated with (per gram total weight) from about 0.01 to about 15 weight percentage of pentosan polysulfate, less than or equal to 30 weight percent water, from about 10 to about 30 weight percent of a lower chain alcohol and a topical carrier selected from the group consisting of propylene glycol, glycerol, glycofurol, poly ethylene glycols (e.g. PEG400, PEG600 and the like), methylparaben, alginates, glyceryl stearate, PEG-100 stearate, cetostearyl alcohol, propylparaben, butylparaben, sorbitols, myristyl alcohol, polyethoxylated anhydrosorbitol monostearate (TWEEN), white soft paraffin (VASELINE), triethanolamine, aloe vera extract, lanolin, cocoa butter or mixtures thereof.

In embodiments, the topical pharmaceutical formulation may also be formulated with (per gram total weight) from about 0.1 to about 10 weight percentage of pentosan polysulfate, less than or equal to 30 weight percent water, from about 10 to about 30 weight percent of a lower chain alcohol and a topical carrier selected from the group consisting of propylene glycol, glycerol, glycofurol, poly ethylene glycols (e.g. PEG400, PEG600 and the like), methylparaben, alginates, glyceryl stearate, PEG-100 stearate, cetostearyl alcohol, propylparaben, butylparaben, sorbitols, myristyl alcohol, polyethoxylated anhydrosorbitol monostearate (TWEEN), white soft paraffin (VASELINE), triethanolamine, aloe vera extract, lanolin, cocoa butter or mixtures thereof.

The formulations of the present disclosure may further comprise additional penetration enhancer(s). The additional penetration enhancer(s) of the present formulation can be selected from a non-limiting group of penetration enhancers known in the art such as fatty acids or fatty acid derivatives, Surfactants (Anionic, cationic or non-ionic surfactants), Azones (such as Lauracapram), Amides (such as Urea and its derivatives), Esters (such as Ethyl acetate, Octyl salicylate), Ethers (such as Dimethyl-isosorbide), Bile salts (such as sodium deoxycholate, sodium taurocholate or sodium glycocholate), Polyols or Glycol derivatives (such as Dipropylene glycol, Monoethyl ether of diethylene glycol) or complex forming agents such as (cyclodextrin or derivatives thereof) etc. The said additional penetration enhancer(s) can be used in an amount ranging from 0 weight percent to 30 weight percent of the formulation In embodiments of the first to fifth aspect, if the treatment is administered by subcutaneous or intravenous injection, the pharmaceutical composition is formulated as an injectable composition.

Currently, there is no cure known for psoriasis. Treatments to relieve psoriasis symptoms typically involve topical treatments, light therapy, and systemic medications and combinations thereof. Most of the known drugs for psoriasis have to be taken regularly for the rest of the patient's life. It is a great advantage of the present disclosure that the effect of the treatment after the course of the treatment may last up to about 4 years before the subject feels the need for another treatment. It is, therefore, an advantage of the present invention that a much smaller total dose of the drug is required to achieve the desired benefits. Furthermore, it is a much more convenient treatment regime than known psoriasis medications.

The pentosan polysulfate, or pharmaceutically acceptable salt thereof, may be administered, for example, until the subject shows partial or complete amelioration of symptoms of psoriasis, psoriatic arthritis and/or dermatitis. Accordingly, the pentosan polysulfate, or pharmaceutically acceptable salt thereof, may be administered repeatedly for a duration from 4 weeks up to any number of weeks.

In embodiments of the first to fifth aspect, the treatment course is once a week over 10 weeks, twice a week over 10 weeks, three times a week over 10 weeks, four times a week over 10 weeks, five times a week over 10 weeks, six times a week over 10 weeks, daily over 10 weeks or twice daily over 10 weeks.

In embodiments of the first to fifth aspect, the treatment course is once a week over 9 weeks, twice a week over 9 weeks, three times a week over 9 weeks, four times a week over 9 weeks, five times a week over 9 weeks, six times a week over 9 weeks, daily over 9 weeks or twice daily over 9 weeks.

In embodiments of the first to fifth aspect, the treatment course is once a week over 8 weeks, twice a week over 8 weeks, three times a week over 8 weeks, four times a week over 8 weeks, five times a week over 8 weeks, six times a week over 8 weeks, daily over 8 weeks or twice daily over 8 weeks.

In embodiments of the first to fifth aspect, the treatment course is once a week over 7 weeks, twice a week over 7 weeks, three times a week over 7 weeks, four times a week over 7 weeks, five times a week over 7 weeks, six times a week over 7 weeks, daily over 7 weeks or twice daily over 7 weeks.

In embodiments of the first to fifth aspect, the treatment course is once a week over 6 weeks, twice a week over 6 weeks, three times a week over 6 weeks, four times a week over 6 weeks, five times a week over 6 weeks, six times a week over 6 weeks, daily over 6 weeks or twice daily over 6 weeks.

In embodiments of the first to fifth aspect, the treatment course is once a week over 5 weeks, twice a week over 5 weeks, three times a week over 5 weeks, four times a week over 5 weeks, five times a week over 5 weeks, six times a week over 5 weeks, daily over 5 weeks or twice daily over 5 weeks.

In embodiments of the first to fifth aspect, the treatment course is once a week over 4 weeks, twice a week over 4 weeks, three times a week over 4 weeks, four times a week over 4 weeks, five times a week over 4 weeks, six times a week over 4 weeks, daily over 4 weeks or twice daily over 4 weeks.

As described in the Examples, it has surprisingly been found that subjects with psoriasis observed great improvements after being treated with pentosan polysulfate, or a pharmaceutically acceptable salt thereof, twice a week for 6 weeks. In a preferred embodiment, the treatment is given twice a week over 6 weeks.

In embodiments of the first to fifth aspect, the pentosan polysulfate, or a pharmaceutically acceptable salt thereof, is in a concentration effective to provide a dose thereof to a subject in an amount of about 0.1 mg/kg body weight to about 4 mg/kg body weight of the subject, or about 0.2 mg/kg to about 4 mg/kg body weight of the subject, or about 0.3 mg/kg to about 4 mg/kg body weight of the subject, or about 0.4 mg/kg to about 4 mg/kg body weight of the subject, or about 0.5 mg/kg to about 4 mg/kg body weight of the subject, or about 0.6 mg/kg to about 4 mg/kg body weight of the subject, or about 0.7 mg/kg to about 4 mg/kg body weight of the subject, or about 0.8 mg/kg to about 4 mg/kg body weight of the subject, or about 0.9 mg/kg to about 4 mg/kg body weight of the subject, or about 1 mg/kg to about 4 mg/kg body weight of the subject.

In embodiments, the pentosan polysulfate, or pharmaceutically acceptable salt thereof, is in a concentration effective to provide a dose thereof to a subject in an amount of about 0.1 mg/kg body weight to about 3 mg/kg body weight of the subject, or about 0.2 mg/kg to about 3 mg/kg body weight of the subject, or about 0.3 mg/kg to about 3 mg/kg body weight of the subject, or about 0.4 mg/kg to about 3 mg/kg body weight of the subject, or about 0.5 mg/kg to about 3 mg/kg body weight of the subject, or about 0.6 mg/kg to about 3 mg/kg body weight of the subject, or about 0.7 mg/kg to about 3 mg/kg body weight of the subject, or about 0.8 mg/kg to about 3 mg/kg body weight of the subject, or about 0.9 mg/kg to about 3 mg/kg body weight of the subject, or about 1 mg/kg to about 3 mg/kg body weight of the subject.

The dose may be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, or 4.0 mg/kg body weight In a preferred embodiment, the pentosan polysulfate, or a pharmaceutically acceptable salt thereof, is in a concentration effective to provide a dose thereof to a subject in an amount of about 2 mg/kg body weight of the subject.

In some embodiments, the pentosan polysulfate, or a pharmaceutically acceptable salt thereof, is administered at a fixed dose from about 25 mg to about 4000 mg. In some embodiments, the fixed dose is about 25 mg, 50 mg, 75 mg, 100 mg, 125 mg, 150 mg, 175 mg, 200 mg, 225 mg, 250 mg, 275 mg, or 300 mg. In some embodiments, the fixed dose is about 350 mg, 400 mg, 450 mg, 500 mg, 550 mg, 600 mg, 650 mg, 700 mg, 750 mg, 800 mg, 850 mg, 900 mg, 950 mg, 1000 mg, 2000 mg, 3000 mg, or 4000 mg.

In embodiments of the first to fifth aspect, if the treatment is administered orally, the pharmaceutical composition is formulated as an oral composition, for example tablets, troches, lozenges, aqueous or oily suspensions, dispersible powders or granules, emulsions, hard or soft capsules, or syrups or elixirs.

If a treatment is to be administered orally, it is preferred that pentosan polysulfate, or a pharmaceutically acceptable salt thereof, is co-administered with a coxib as disclosed in PCT/AU2019/050119, the contents of which are included by way of cross-reference and in particular as disclosed at page 26, lines 1-9.

In embodiments of the first to fifth aspect, the pentosan polysulfate, or a pharmaceutically acceptable salt thereof, is in a concentration effective to provide a dose thereof to a subject in an amount of about 0.1 mg/kg body weight to about 4 mg/kg body weight of the subject, or about 1 mg/kg to about 4 mg/kg body weight of the subject, or about 1 mg/kg to about 3 mg/kg body weight of the subject. The treatment course may be once a week over 8 weeks, twice a week over 8 weeks, three times a week over 8 weeks, four times a week over 8 weeks, five times a week over 8 weeks, six times a week over 8 weeks, daily over 8 weeks or twice daily over 8 weeks. The treatment course may be once a week over 7 weeks, twice a week over 7 weeks, three times a week over 7 weeks, four times a week over 7 weeks, five times a week over 7 weeks, six times a week over 7 weeks, daily over 7 weeks or twice daily over 7 weeks. The treatment course may be once a week over 6 weeks, twice a week over 6 weeks, three times a week over 6 weeks, four times a week over 6 weeks, five times a week over 6 weeks, six times a week over 6 weeks, daily over 6 weeks or twice daily over 6 weeks. Once the treatment has achieved an appropriate reduction in the subject's symptoms, a maintenance dose may be administered by a topical application. Such a dose may be in the range of 0.01 mg/kg body weight to about 0.36 mg/kg body weight.

In embodiments, pentosan polysulfate, or a pharmaceutically acceptable salt thereof, may be co-administered with drugs that are commonly used to treat psoriasis, psoriatic arthritis and/or dermatitis.

In embodiments of the first to fifth aspect, the subject commences treatment with pentosan polysulfate, or a pharmaceutically acceptable salt thereof, when other treatments for psoriasis, psoriatic arthritis and/or dermatitis have failed to produce the desired clinical outcome.

It will be appreciated that the specific dose and frequency of dosage for any particular patient may be varied and can depend upon a variety of factors including the patient's response to the PPS, or a pharmaceutically acceptable salt thereof, the metabolic stability and length of action of the PPS, the age, body weight, general health, sex, diet, mode and time of administration, rate of excretion, drug co-administration, the severity of the particular condition, and the host undergoing therapy.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### Experimental

### Measurement of PPS Polydispersity (PDI)

The NaPPS used in the examples was purchased from Bene-PharmaChem GmbH & Co KG, Geretsried, Germany. The material was used without any further purification or fractionation (i.e. it is unfractionated PPS) and batches will typically have a molecular weight of 4000 to 6000 Da. The PDI of the PPS samples is typically around 2.1 when measured by a size-exclusion chromatography (SEC)—Refractive Index (RI) approach or around 1.5-1.7 when measured using a size-exclusion chromatography (SEC)—Triple Detection (TD) approach.

The PDI may be obtained by a number of approaches which are known in the field. The two main accepted approaches, which provide for different PDI values, are described below.

### Size-Exclusion Chromatography (SEC)—Refractive Index (RI)

The supplied PPS is suspended in 30 ml deionized water and then centrifuged at 3500 rpm (1370 g) for 15 min. The supernatant was separated and subsequently passed twice through a 0.20 μm syringe filter (Chromafil® Xtra, Macherey-Nagel, Germany). The resulting clear solution was freeze-dried to obtain the PPS for analysis. The molecular weight determination was conducted on a GPC-System (Malvern Panalytical, United Kingdom) with refractive index (RI) detection, using the following method: Columns (PSS GmbH, Germany): MCX 5 μm, 1000 Å guard column, MCX 5 μm, 8×300 mm, 1000 Å, MCX 5 μm, 8×300 mm, 100 Å connected in series. Mobile phase: deionized water containing 0.9 % NaCl (w/w) and 0.02 % sodium azide (w/w). Flowrate: 0.65 ml/min. Temperature of columns and detectors: 45° C. Sample concentration: 40 mg/ml in mobile phase. Injection volume: 40 μl. dn/dc: 0.1098.

### Size-Exclusion Chromatography (SEC)—Triple Detection (TD)

The PDI may be determined using the approach detailed in the following article which is herein incorporated by reference in its entirety: Alekseeva A, et al. In-depth structural characterization of pentosan polysulfate sodium complex drug using orthogonal analytical tools. Carbohydr. Polym. 2020 April 15; 234:115913. doi: 10.1016/j.carbpol.2020.115913. Epub 2020 Jan. 27. PMID: 32070534; PMCID: PMC7171972. Briefly, the PPS sample is subjected to a water extraction procedure to separate PPS material from the insoluble cellulose component. The PPS from a capsule (about 250 mg) is suspended in 10 ml of water and stirred at room temperature for 16 h. Filtration can be carried out using a mixed cellulose esters membrane with 0.22 μm cut-off (Millipore GSWP04700). The water-soluble portion of each sample can be freeze dried to give the PPS for analysis. Measurements for molecular weight distribution can be performed on a Viscotek 305 HPLC system (Houston, Texas) equipped with a triple detector array using simultaneous action of a refraction index detector (RI), viscometer and Right Angle Laser Light-scattering (RALLS) detector. To isolate size specific fractions of PPS about 120 mg of water soluble PPS material can be dissolved in 2.5 ml of water and loaded onto a chromatographic Knauer Bioline system equipped with two Superdex S30 columns (48cm×3 cm, and 83 cm×2 cm, GE Healthcare Life Sciences) and 0.25 M ammonium chloride as eluent. Separation can be performed at room temperature using a flow-rate of 5 ml/min. UV detection at 260 nm may be used to monitor the fractionation profile. Each fraction which is obtained can be desalted on a Biofox 40/100 SEC Agarose column (3.0 cm×90 cm, Knauer) using 10% aqueous ethanol with a flow-rate of 15 ml/min and UV monitoring at 210 nm.

Alternatively, the approach in the following article also uses a triple detection array and will provide similar PDI values to the above Alekseeva methodology. This approach may also be used to determine the PDI of samples for use in the treatment of the present disclosure and the following reference is herein incorporated by reference in its entirety: Dominik Lenhart, et al. Chemical and biological differences between original and mimetic pentosan polysulfates; Carbohydrate Polymers, Volume 319, 2023, 121201, ISSN 0144-8617; https://doi.org/10.1016/j.carbpol.2023.121201.

### Example 1

Patient JC, a female around 61 years of age, presented for management of knee arthritis in January of 2018. She was treated with pentosan polysulfate in June of 2018. The treatment regimen consisted of administration of PPS, at a preferred dose of 2 mg/kg of bodyweight, twice per week for 6 weeks by subcutaneous injections (total of 12 injections).

At that time she had cutaneous psoriasis involving the scalp and face. This had been chronic and unrelenting despite other therapies. She would use her hair to cover the unsightly and pruritic rash on her forehead. At that time she was not taking any other medication for her psoriasis.

JC reported post treatment with pentosan polysulfate that the scalp and face psoriasis resolved and that this had not happened with other previous treatments. There was no obvious psoriasis and this initial report was 4 months post administration of the pentosan polysulfate.

To mid-2023 she has reported sustained relief of symptoms of the psoriasis.

### Example 2

Patient AD, a male around 71 years of age, presented for management of osteoarthritis of both knees. His initial consultation was on 18 Jan. 2018.

His past history includes chronic cutaneous psoriasis that involves the torso and limbs. The psoriasis has been poorly responsive to multiple different therapies.

He had diffuse psoriatic rash at time of my initial consultation manifest as a red scaling rash.

His medications were naproxen, omeprazole and aspirin.

AD underwent treatment with pentosan polysulfate by twice weekly subcutaneous injections at a dose of 2 mg per kg for a total of 6 weeks for osteoarthritis.

At post treatment review in May of 2018 AD reported that his psoriasis had significantly improved both in extent and symptoms. The improvement endured for a period of approximately six months and, afterwards, his psoriasis only came back mildly. His psoriasis is normally unrelenting.

1. A method of treating a subject suffering from psoriasis comprising the steps of:
(i) determining that the subject is unacceptably responsive to one or more treatments, and
(ii) administering to the subject a subcutaneous dose of a sodium pentosan polysulfate (NaPPS), having an average molecular weight of 4000 to 6000 Daltons and a PDI of about 2.1, in an amount of 2 mg/kg body weight of the subject twice weekly for six weeks.

2. (canceled)

3. (canceled)

4. The method of claim 1, wherein the psoriasis is selected from the group consisting of plaque psoriasis, guttate psoriasis, pustular psoriasis, inverse psoriasis and erythrodermic psoriasis.

5. The method claim of claim 1, wherein the subject is a mammal.

6. The method of claim 1, wherein the subject is a human subject.
